(19) United States
(12) Patent Application Publication
(10) Pub. No.: US 2025/0204750 A1
(43) Pub. Date: Jun. 26, 2025

(54) ENDOSCOPIC TREATMENT EQUIPMENT

(71) Applicant: FUJIFILM Corporation, Tokyo (JP)

(72) Inventor: Yoshihiro UEDA, Ashigarakami-gun (JP)

(73) Assignee: FUJIFILM Corporation, Tokyo (JP)

(21) Appl. No.: 18/976,342

(22) Filed: Dec. 11, 2024

Related U.S. Application Data
(60) Provisional application No. 63/613,767, filed on Dec. 22, 2023.

(51) Int. Cl. A61B 1/00

(52) U.S. Cl. CPC A61B 1/00087; A61B 1/00128; A61B 1/00133

(57) ABSTRACT

An endoscopic treatment equipment to be introduced into a treatment tool inlet port of an endoscope, includes: a distal end portion that includes a treatment part; a proximal end portion that is provided at a proximal end of the endoscopic treatment equipment; an operation member that is provided between the distal end portion and the proximal end portion and that is mechanically connected to the treatment part; a first connecting member that connects the distal end portion and the operation member; and a second connecting member that connects the operation member and the proximal end portion and that is different from the first connecting member, the operation member is configured to be rotationally moved in a first direction about an axis of the endoscopic treatment equipment, and the treatment part is configured to be rotationally moved in the first direction by a rotational movement of the operation member.

### CROSS REFERENCE TO RELATED APPLICATION

This application is based on and claims priority from U.S. Provisional Application No. 63/613,767 filed on Dec. 22, 2023, the entire disclosure of which is incorporated herein by reference.

### BACKGROUND OF THE INVENTION

### 1. Field of the Invention

The present disclosed technology relates to endoscopic treatment equipment.

### 2. Description of the Related Art

WO2017/86107A describes a treatment tool inserting tool that is attached to an insertion part of an inserting device inserted into a subject and that is for inserting a treatment tool into the insertion part, the treatment tool inserting tool having a body part into which the treatment tool is inserted, an attachment part for attaching the body part to the insertion part, a guide unit that is connectable to a treatment tool insertion port of the inserting device and that is for guiding the treatment tool which extends from the body part into the insertion part via the treatment tool insertion port, and an operation member that is provided at the body part, that is capable of fixing the treatment tool inserted into the body part as at least a part thereof elastically deforms, and that is for performing an operation of advancing and retracting of the treatment tool along an axial direction and rotationally moving the treatment tool about an axis in a fixed state where the treatment tool is fixed.

JP2005-198868A describes an endoscope treatment tool comprising a treatment tool insertion part that is insertable into a channel of an endoscope, a treatment part that is connected to a distal end of the treatment tool insertion part, and a treatment tool operating part that is disposed on a proximal end side of the treatment tool insertion part, that integrally has a rotation operating part which is capable of rotating the treatment tool insertion part about an axis and an advancing and retracting operating part which is capable of advancing and retreating the treatment tool insertion part in an axial direction, and that is attachable to and detachable from the endoscope.

JP2008-148738A describes an endoscope treatment tool comprising a movable distal end portion that has a movable portion and a fixing unit and that treats a living body, a single coil sheath around which one solid wire is helically wound, a braided sheath, in which a plurality of thin wires are braided and a resin is infused in a knot, and which is wrapped around the single coil sheath, an operation shaft member that is formed to extend in an elongated shape and that has a distal end connected to the movable portion, and an operating part that operates the operation shaft member so that the operation shaft member advances and retracts, in which a distal end of the braided sheath is connected to the fixing unit, and a proximal end is connected to the operating part.

JP2014-4333A describes a high-frequency treatment device that injects a saline solution and that raises a lesion tissue in order to separate the lesion tissue from a normal tissue and then removes the lesion tissue through scraping using high frequencies. In the high-frequency treatment device, in front of an operating tool that moves forward and backward along a tubular body provided with a saline solution injection port on the front and that has a high-frequency port, a push rod is bonded to be positioned inside the body, an auxiliary push rod that has a locking surface on an outer peripheral surface thereof is rotatably bonded to a front end of the push rod of the operating tool, a handle that has a locking corresponding hole locked with the locking surface of the auxiliary push rod is engaged with a front side portion of the body non-attachably and non-detachably, an operation unit is configured such that a wire coupled to a front end of the auxiliary push rod is exposed via a tube bonded in front of the body, an arc-shaped knife is bonded to an end portion of the wire of the operation unit, the push rod, the auxiliary push rod, the wire, and the knife are configured to operate to advance and retract in response to an advancing and retracting operation, and the wire and the knife are configured to rotate in response to the rotation of the handle.

JP1997-299323A (JP-H-9-299323A) describes a treatment tool guiding tool of an endoscope in which a flexible tubular member that is freely inserted into and removed from a treatment tool insertion channel of the endoscope, which is a portion protruding outward from a distal end of the treatment tool insertion channel, is formed to be curved in an arc shape.

JP2010-22619A describes an endoscope treatment tool that is provided with, at a distal end of a flexible cord which passes through a treatment tool insertion channel from a treatment tool outlet port of an endoscope and which has a length allowing it to be led out from a treatment tool inlet port, a treatment function unit which is operable in response to an operation of an operation handle unit of which a rear end side is coupled to the flexible cord, and in which the treatment function unit has directivity in a rotation direction. In the endoscope treatment tool, the flexible cord has a length to be at a position on a proximal end side of the treatment tool outlet port in a state where the treatment function unit is led out from the treatment tool inlet port, a rotational operation member is provided to be fixed to an outer peripheral portion at a position where the flexible cord is led out from the treatment tool outlet port to the outside, and direction adjustment of the treatment function unit is configured to be possible by rotating the rotational operation member and rotating the flexible cord.

### SUMMARY OF THE INVENTION

In the present disclosure, endoscopic treatment equipment that can be used by a plurality of persons in collaboration (used together at the same timing) is provided.

According to an aspect of the present disclosed technology, there is provided endoscopic treatment equipment to be used by being introduced into a treatment tool inlet port of an endoscope, the endoscopic treatment equipment comprising a distal end portion that includes a treatment part, a proximal end portion that is provided at a proximal end, an operation member that is provided between the distal end portion and the proximal end portion and that is mechanically connected to the treatment part, a first connecting member that connects the distal end portion and the operation member, and a second connecting member that connects the operation member and the proximal end portion and that is different from the first connecting member, in which the operation member is configured to be rotationally moved in a first direction about an axis of the endoscopic treatment equipment, and the treatment part is configured to be rotationally moved in the first direction by a rotational movement of the operation member.

According to another aspect of the present disclosed technology, there is provided endoscopic treatment equipment to be used by being introduced into a treatment tool inlet port of an endoscope, the endoscopic treatment equipment comprising a distal end portion that includes a treatment part, a proximal end portion that is provided at a proximal end, a first member that extends from the distal end portion to the proximal end portion along an axial direction of the endoscopic treatment equipment, and a tubular member in which a part of the first member is provided and that extends from the distal end portion to a position between the distal end portion and the proximal end portion, in which the treatment part is configured to be rotationally moved in a first direction about an axis of the endoscopic treatment equipment by a rotational movement of the tubular member in the first direction.

With the present disclosed technology, as an operator performs an operation requiring skill, and an assistant performs other simple operations, a treatment can be efficiently and accurately performed.

### BRIEF DESCRIPTION OF THE DRAWINGS

FIG. 1 is a view showing a schematic configuration of an endoscope device 100 which is an aspect of the present disclosed technology.

FIG. 2 is a schematic view showing a schematic configuration of endoscopic treatment equipment 20 that is an embodiment of endoscopic treatment equipment to be used by being inserted into a treatment tool inlet port 112 of an endoscope 1.

FIG. 3 is a view showing a state where the endoscopic treatment equipment 20 shown in FIG. 2 is introduced into the treatment tool inlet port 112 of the endoscope 1.

FIG. 4 is an enlarged perspective view of a vicinity of a connector portion 22 in the endoscopic treatment equipment 20.

FIG. 5 is an enlarged perspective view of a vicinity of a distal end portion 21 of the endoscopic treatment equipment 20.

FIG. 6 is a cross-sectional view of the vicinity of the distal end portion 21 shown in FIG. 5.

FIG. 7 is a schematic cross-sectional view of a vicinity of an operation handle 23 in the endoscopic treatment equipment 20.

FIG. 8 is a schematic view of a cross section of the operation handle 23 perpendicular to an axial direction and is a schematic view of a cross section through a flat pipe 234.

FIG. 9 is a view showing a state where a support member 231 is rotationally moved from the state of FIG. 8.

FIG. 10 is a perspective view showing a configuration of an operation handle 23A that is a first modification example of the operation handle 23.

FIG. 11 is a schematic cross-sectional view of the operation handle 23A shown in FIG. 10.

FIG. 12 is a schematic cross-sectional view showing a configuration of an operation handle 23B that is a second modification example of the operation handle 23.

FIG. 13 is a schematic cross-sectional view taken along line A-A of FIG. 12.

FIG. 14 is a view showing a state where a support member 231C is rotationally moved from the state of FIG. 13.

FIG. 15 is a schematic view showing a configuration of endoscopic treatment equipment 20A that is a first modification example of the endoscopic treatment equipment 20.

FIG. 16 is a schematic cross-sectional view showing a configuration of an operation handle 23D that is a third modification example of the operation handle 23 of the endoscopic treatment equipment 20.

FIG. 17 is a schematic view showing a configuration of endoscopic treatment equipment 20E that is a second modification example of the endoscopic treatment equipment 20.

FIG. 18 is a schematic view showing a cross section of a vicinity of an operation handle 23E of the endoscopic treatment equipment 20E shown in FIG. 17.

### DESCRIPTION OF THE PREFERRED EMBODIMENTS

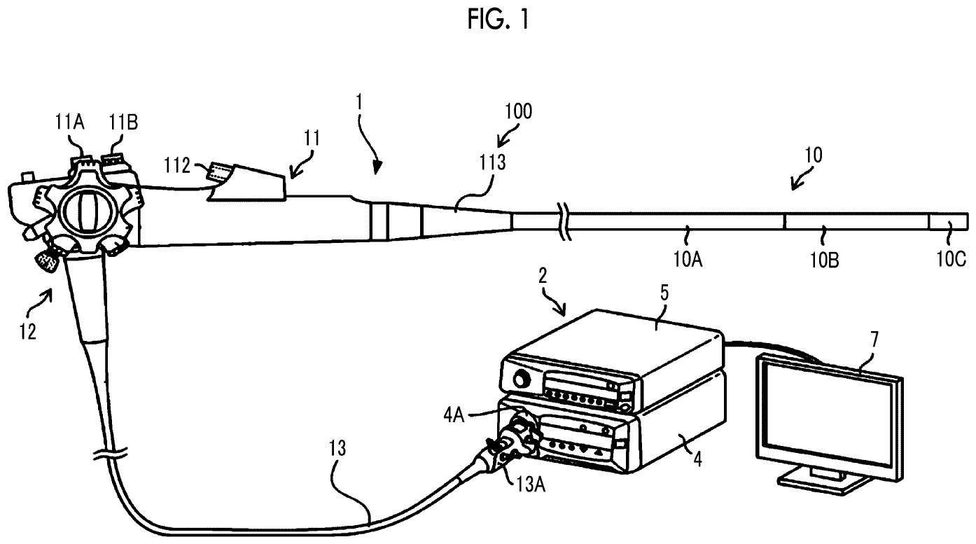

FIG. 1 is a view showing a schematic configuration of an endoscope device 100 which is one aspect of the present disclosed technology. The endoscope device 100 comprises an endoscope 1, a body part 2 consisting of a light source device 4 and a processor device 5 to which the endoscope 1 is connected, and a display device 7 that displays a captured image and the like obtained by imaging with the endoscope 1.

The endoscope 1 comprises an insertion part 10 that is an elongated instrument extending in one direction and that is to be inserted into a subject, an operating part 11 that is provided at a proximal end portion of the insertion part 10 and that is provided with an operation member for performing an observation mode switching operation, an imaging and storing operation, a forceps operation, an air/water supply operation, a suction operation, an electric scalpel operation, or the like, an angle knob 12 that is provided adjacent to the operating part 11, and a universal cord 13 including a connector portion 13A that attachably and detachably connects the endoscope 1 to a connecting portion 4A of the light source device 4.

The operating part 11 is provided with a treatment tool inlet port 112 through which endoscopic treatment equipment is introduced. The endoscopic treatment equipment is an elongated instrument longer than the insertion part 10 of the endoscope 1, and a treatment part is provided at a distal end thereof. A device to which the present disclosed technology can be preferably applied is a device that can rotationally move the treatment part about an axis of the endoscopic treatment equipment. The endoscopic treatment equipment is, for example, biopsy forceps, grasping forceps, a hemostatic clip, a scissors type knife, a snare, an injection needle, or a high-frequency knife. Although not shown in FIG. 1, inside the operating part 11 and the insertion part 10, various types of channels, such as a treatment tool channel, an air/water supply channel, and a suction channel, through which the endoscopic treatment equipment introduced from the treatment tool inlet port 112 is inserted, are provided. The operating part 11 includes a suction button 11A, an air/water supply button 11B, and the like.

The insertion part 10 is composed of a flexible soft portion 10A, a bendable part 10B provided at a distal end of the soft portion 10A, and a distal end portion 10C that is harder than the soft portion 10A and that is provided at a distal end of the bendable part 10B. An imaging element and an imaging optical system are built in the distal end portion 10C.

The bendable part 10B is configured to bend via a rotational movement operation of the angle knob 12. The bendable part 10B can be bent in any direction and at any angle depending on a part of the subject or the like for which the endoscope 1 is used, and the distal end portion 10C can be directed in a desired direction.

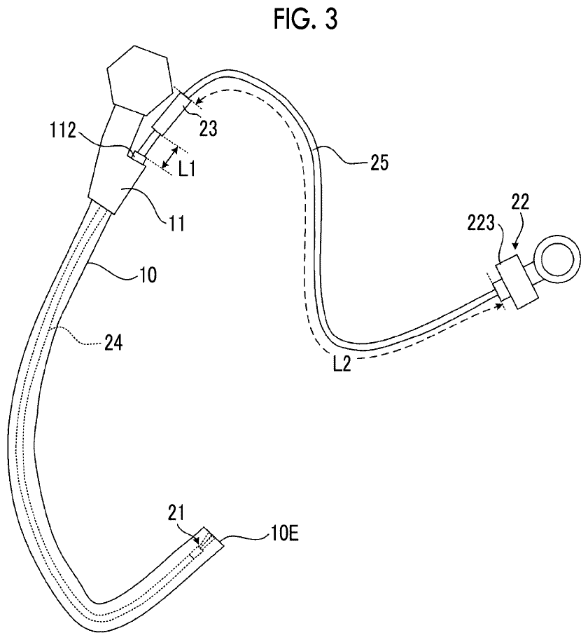

Inside the endoscope 1, a light guide composed of a plurality of optical fibers bundled from the distal end portion 10C of the insertion part 10 to the connector portion 13A is provided. Light generated by the light source device 4 is introduced into the light guide from the connector portion 13A, advances to the distal end portion 10C, and is emitted to the subject from an illumination window provided at the distal end portion 10C. The treatment tool channel is provided inside the endoscope 1 from the distal end portion 10C of the insertion part 10 to the treatment tool inlet port 112. A distal end of the endoscopic treatment equipment introduced into the treatment tool inlet port 112 can protrude from a treatment tool opening provided in a distal end surface 10E (see FIG. 3) of the distal end portion 10C.

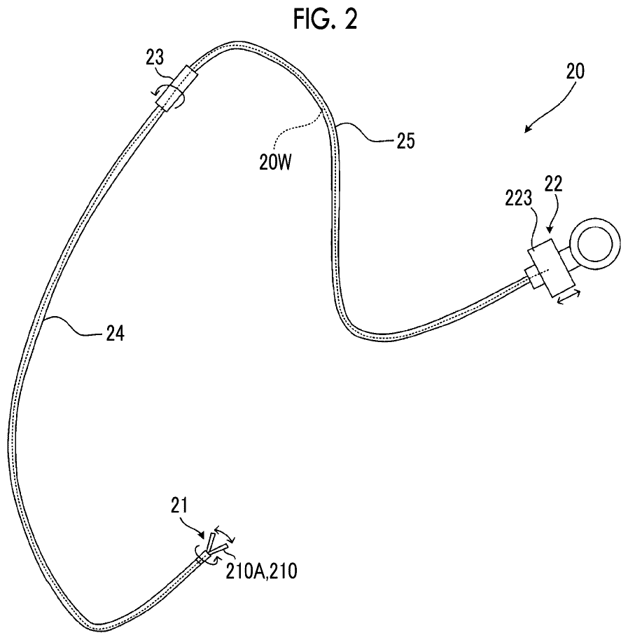

FIG. 2 is a schematic view showing a schematic configuration of endoscopic treatment equipment 20 that is an embodiment of the endoscopic treatment equipment to be used by being inserted into the treatment tool inlet port 112 of the endoscope 1. The endoscopic treatment equipment 20 comprises a distal end portion 21 including a treatment part 210, a connector portion 22 provided at a proximal end, an operation handle 23 that is provided between the distal end portion 21 and the connector portion 22 and that is mechanically connected to the treatment part 210, a first sheath 24 that connects the distal end portion 21 and the operation handle 23, and a second sheath 25 that connects the operation handle 23 and the connector portion 22 and that is different from the first sheath 24. The connector portion 22 constitutes a proximal end portion. The operation handle 23 constitutes an operation member. The first sheath 24 constitutes a first connecting member. The second sheath 25 constitutes a second connecting member.

The connector portion 22 has a slider 223 that is movable in an axial direction of the endoscopic treatment equipment 20 (hereinafter, simply referred to as an axial direction, which means the axial direction of the endoscopic treatment equipment 20 (in other words, a longitudinal direction)). For example, the connector portion 22 is provided with a connector for electrically connecting to the endoscopic treatment equipment 20 inside the slider 223. The treatment part 210 has a pair of forceps pieces 210A, and the pair of forceps pieces 210A are configured to be opened and closed.

Each of the first sheath 24 and the second sheath 25 has an elongated columnar shape. Each of the first sheath 24 and the second sheath 25 preferably has an elongated tubular shape and has flexibility. It is preferable that the length of the first sheath 24 is longer than a distance from the distal end surface 10E of the insertion part 10 to an inlet of the treatment tool inlet port 112.

The operation handle 23 has a tubular shape, the first sheath 24 is connected to one end side thereof in the axial direction, and the second sheath 25 is connected to the other end side thereof in the axial direction. As described above, inner spaces of the first sheath 24 and the second sheath 25 are configured to be connected to each other via the operation handle 23. Since the operation handle 23 is a portion operated by an operator who operates the endoscope 1, it is preferable that the operation handle 23 is configured to have higher stiffness than stiffness of the first sheath 24 and stiffness of the second sheath 25 in consideration of operability and durability. In addition, it is preferable that an outer diameter of the operation handle 23 is larger than an inner diameter of the treatment tool inlet port 112 so that the operation handle 23 is not inserted into the treatment tool inlet port 112 and is easily operated. In order to make the operation easier, it is also effective to provide a configuration where an outer peripheral surface of the operation handle 23 is provided with unevenness or the like to prevent slipping.

An operation wire 20W is provided in the inner spaces of the first sheath 24, the operation handle 23, and the second sheath 25. The operation wire 20W is provided to extend from the connector portion 22 to the treatment part 210 and mechanically connects the slider 223 of the connector portion 22 and the treatment part 210. The operation wire 20W is configured to be moved in the axial direction in accordance with the movement of the slider 223. In the endoscopic treatment equipment 20, by performing an operation of moving the slider 223 in the axial direction, the pair of forceps pieces 210A provided in the treatment part 210 can be opened and closed via the operation wire 20W. The operation wire 20W constitutes a transmission element (hereinafter, also referred to as a power transmission element) that transmits power corresponding to the movement of the slider 223 to the treatment part 210.

The position of the operation handle 23 in the axial direction is fixed to the first sheath 24 and the second sheath 25. That is, in a case where the operation handle 23 is grasped and moved in the axial direction, the first sheath 24 and the second sheath 25 move in the axial direction together with the operation handle 23.

Meanwhile, the operation handle 23 is configured to be rotationally moved with respect to the first sheath 24 and the second sheath 25 in a first direction about the axis of the endoscopic treatment equipment 20. The operation wire 20W is configured to be moved in the axial direction with respect to the operation handle 23 and is configured not to be moved rotationally in the first direction. That is, in a case where the operation handle 23 is moved rotationally in the first direction with respect to the first sheath 24 and the second sheath 25, the operation wire 20W is also configured to be moved rotationally in the first direction in conjunction with the rotational movement of the operation handle 23. As described above, by rotationally moving the operation handle 23, the treatment part 210 connected to a distal end of the operation wire 20W can be moved rotationally in the first direction in conjunction with the rotational movement.

FIG. 3 is a view showing a state where the endoscopic treatment equipment 20 shown in FIG. 2 is introduced into the treatment tool inlet port 112 of the endoscope 1. The endoscopic treatment equipment 20 is introduced from the treatment tool inlet port 112 and is led out from the distal end surface 10E via a treatment tool insertion channel (not shown). In the state shown in FIG. 3, positions of the distal end of the treatment part 210 and the distal end surface 10E in the axial direction match each other, and a part of the first sheath 24 on a proximal end side is exposed outside the treatment tool inlet port 112. In the state shown in FIG. 3, the length of the exposed portion of the first sheath 24 (a distance between the treatment tool inlet port 112 and the operation handle 23) is a distance L1.

The endoscopic treatment equipment 20 can be operated by the operator who operates the endoscope 1 alone, but can also be operated by two persons including the operator and an assistant who assists in the operation of the endoscopic treatment equipment 20 in cooperation. Specifically, the operator grasps the operating part 11 with his or her left hand and pinches the operation handle 23 with his or her right hand fingers to perform an advancing and retracting operation of the operation handle 23 (movement in the axial direction) and a rotational movement operation of the operation handle 23 (rotational movement in the first direction).

The operator can make the treatment part 210 protrude from the distal end surface 10E by the distance L1 at most by performing the advancing and retracting operation of the operation handle 23. In addition, the operator can adjust orientations of the forceps pieces 210A by performing the rotational movement operation of the operation handle 23. The assistant moves the slider 223 of the connector portion 22 to perform an opening and closing operation of the forceps pieces 210A.

As described above, with the endoscopic treatment equipment 20, the operator can perform work requiring skill (direction determination and positioning of the forceps pieces 210A with respect to a treatment target part), and the assistant can perform simple work not requiring that much skill (treatment of the treatment target part by opening and closing the forceps pieces 210A). As described above, it is possible to efficiently and accurately perform the treatment by dividing the roles. By using the endoscopic treatment equipment 20, the assistant only needs to move the slider 223 in accordance with an instruction of the operator. For this reason, an efficient treatment is possible regardless of a combination of the operator and the assistant.

A distance L2 from the operation handle 23 to the connector portion 22 is preferably, for example, 50 cm or more in order to ensure an appropriate distance between the operator and the assistant. The distance L1 may be a distance at which the operator can easily perform an operation, and an upper limit value thereof is preferably, for example, 30 cm. In many types of endoscopic treatment equipment, a protrusion length from the distal end surface 10E is in a range of 5 cm or more and 10 cm or less. Therefore, it is preferable that the distance L1 is set to be in this range.

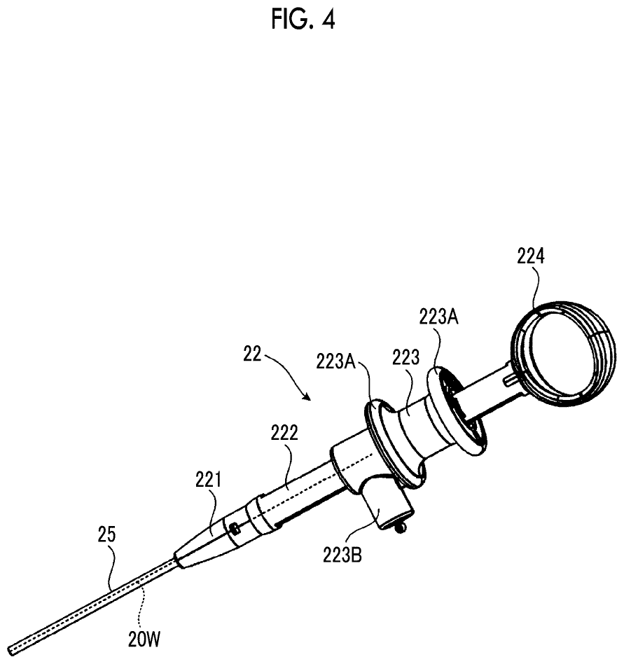

FIG. 4 is an enlarged perspective view of a vicinity of the connector portion 22 in the endoscopic treatment equipment 20. The connector portion 22 comprises a tubular sheath support portion 221 that supports a proximal end portion of the second sheath 25 as the proximal end portion thereof is fixed inside, a slider shaft 222 that has a distal end fixed to the sheath support portion 221, the slider 223 that is supported to be slidable in the axial direction on an outer periphery of the slider shaft 222, a ring 224 that is provided at a proximal end of the slider shaft 222, and a cord coupling part 223B that is provided at the slider 223.

A cord connected to a control device of the endoscopic treatment equipment 20 is coupled to the cord coupling part 223B. In a case where the endoscopic treatment equipment 20 is electrically connectable to the treatment part 210, electrical connection to the treatment part 210 is performed via the cord coupling part 223B. In this case, an electric wire electrically connected to the treatment part 210 is provided inside the first sheath 24, the operation handle 23, and the second sheath 25 to extend into the slider shaft 222, and the electric wire is connected to a connector inside the cord coupling part 223B. This electric wire constitutes a transmission element (hereinafter, also referred to as a current transmission element) that transmits a high-frequency current from the connector portion 22 to the treatment part 210. In a case where the endoscopic treatment equipment 20 is not electrically connected to the treatment part 210, the electric wire and the cord coupling part 223B can be omitted.

For example, in a case where the present disclosed technology is applied to a snare, the treatment part 210 is configured to be substituted with an annular or bag-shaped snare wire of which a shape changes in accordance with the movement of the slider 223 in the endoscopic treatment equipment 20.

In addition, for example, in a case where the present disclosed technology is applied to an injection needle, the treatment part 210 is substituted with a cylindrical needle in the endoscopic treatment equipment 20, a coupling part of a cylinder for supplying a chemical liquid is added to the connector portion 22, and a flow passage for passing the chemical liquid is configured to be provided in the connector portion 22, the second sheath 25, the operation handle 23, the first sheath 24, and the distal end portion 21. This flow passage constitutes a transmission element (hereinafter, also referred to as a chemical liquid transmission element) that transmits the chemical liquid. For example, in a case where the treatment part 210 is a curved needle, the operator can change the orientation of the needle by rotationally moving the operation handle 23. In this case, the assistant may perform only injection work of the chemical liquid by operating the cylinder in accordance with an instruction of the operator.

A space where the operation wire 20W is movable in the axial direction is provided inside the slider shaft 222, and a wire support member (not shown) that supports a proximal end of the operation wire 20W is provided in the space. The wire support member is fixed to the slider 223 and moves in conjunction with the slider 223.

For example, in a state where the assistant's thumb is hooked to the ring 224 and his or her index finger and middle finger are hooked between two flange portions 223A provided in the slider 223, the assistant pulls his or her index finger and middle finger to a thumb side to move the slider 223 in the axial direction. In conjunction with this movement, the wire support member is moved in the axial direction, so that the operation wire 20W is moved in the axial direction.

It is preferable that the wire support member built in the slider shaft 222 supports the operation wire 20W to be movable rotationally in the first direction. That is, it is preferable that the operation wire 20W and the connector portion 22 are connected to be rotationally movable relative to each other in the first direction. In this manner, a rotational movement force of the operation handle 23 can be prevented from being transmitted to the connector portion 22 via the operation wire 20W. Since the operation handle 23 can be moved rotationally without any problem even in a state where the assistant holds the connector portion 22, workability of the operator and the assistant can be improved.

In a configuration where the wire support member supports the operation wire 20W to be not movable rotationally, a rotational movement force is transmitted to the connector portion 22 via the operation wire 20W in a case where the operation handle 23 is rotationally moved. However, even in this case, the assistant changes the way of holding the connector portion 22 or the operation wire 20W is twisted without opposing the rotational movement force, so that the operation handle 23 can be moved rotationally without any problem. As will be described later, by setting at least a part of the operation wire 20W in a range where the operation handle 23 and the connector portion 22 are connected to have low rotational movement direction stiffness, the rotational movement force transmitted to the connector portion 22 can be reduced.

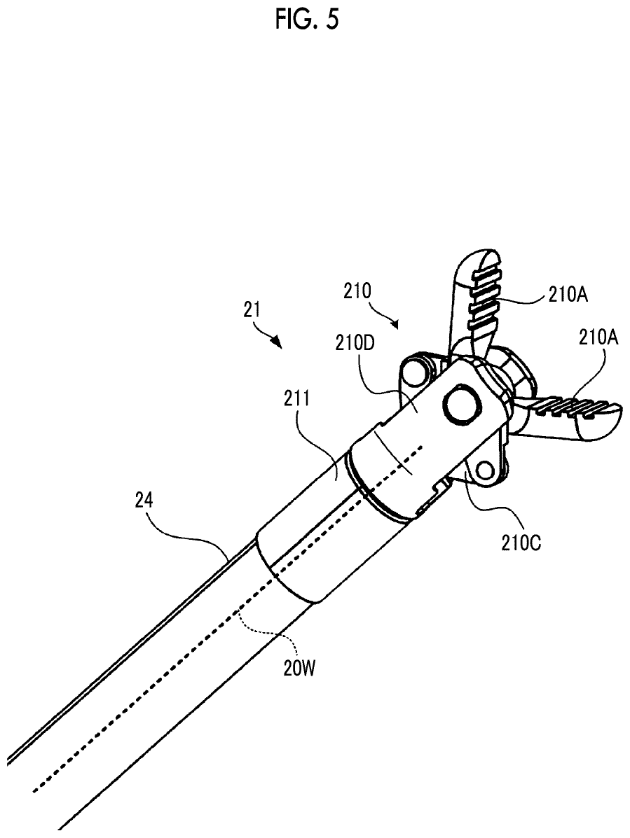

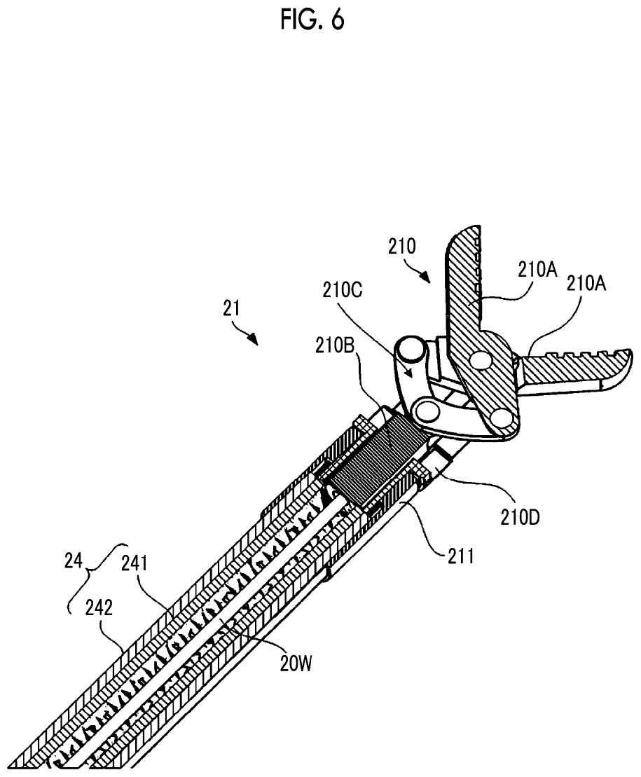

FIG. 5 is an enlarged perspective view of a vicinity of the distal end portion 21 of the endoscopic treatment equipment 20. FIG. 6 is a cross-sectional view of the vicinity of the distal end portion 21 shown in FIG. 5. The distal end portion 21 comprises a distal end support member 211 and the treatment part 210. The distal end support member 211 has a tubular shape and supports a distal end portion of the first sheath 24 inside on the proximal end side.

The treatment part 210 comprises a wire support member 210B that is provided to be movable in the axial direction and that supports the distal end of the operation wire 20W inside on a distal end side of the distal end support member 211, a link mechanism 210C that couples the wire support member 210B and the forceps pieces 210A, and a link mechanism support member 210D that supports the link mechanism 210C. The wire support member 210B and the link mechanism support member 210D are supported by the distal end support member 211 to be movable rotationally in the first direction.

In a case where the operation wire 20W is rotationally moved and the wire support member 210B is rotationally moved, the link mechanism 210C, the forceps pieces 210A, and the link mechanism support member 210D are rotationally moved with respect to the distal end support member 211 in conjunction with the wire support member 210B. As described above, in the endoscopic treatment equipment 20, the operation handle 23 and the treatment part 210 are mechanically connected by the operation wire 20W, and a rotational movement force in a case where the operation handle 23 is rotationally moved in the first direction is configured to be transmitted to the treatment part 210 via the operation wire 20W.

As shown in FIG. 6, the first sheath 24 preferably has a two-layer structure having a first tubular member 241 and a second tubular member 242 provided on an outer periphery of the first tubular member 241. The operation wire 20W is provided inside the first tubular member 241 to be movable in the axial direction and rotationally movable in the first direction. The first tubular member 241 is composed of, for example, a closely attached coil spring. The second tubular member 242 is composed of, for example, a net blade tube, a multi-layer coil, a resin tube formed by extrusion forming, or the like. In the first sheath 24, the second tubular member 242 may be omitted.

While the grasping forceps or hemostatic forceps generate a relatively strong wire pulling force in a case of closing a forceps piece, the distal end of the treatment part 210 needs to be accurately positioned with respect to a lesion. For this reason, a characteristic of a sheath in which a change in length is unlikely to occur with respect to compression (force in the axial direction) is required for the first sheath 24. In addition, since the first sheath 24 is a portion to be inserted into the insertion part 10, durability and insertability are also required. The second sheath 25 is sufficient insofar as the operation wire 20W can be accommodated to be movable in at least the axial direction, unlike the first sheath 24. For this reason, the second sheath 25 may have a characteristic in which a length changes greatly with respect to compression, and durability and insertability are not required much. Therefore, the second sheath 25 may be composed of, for example, only a single inexpensive member such as a resin tube formed by extrusion forming. As described above, as the first sheath 24 and the second sheath 25 are separated from each other, it is easy to change mechanical properties between the first sheath 24 and the second sheath 25.

For example, it is preferable that the first sheath 24 has higher stiffness against a force in the axial direction than stiffness of the second sheath 25. In this manner, manufacturing costs of the second sheath 25 can be lowered while ensuring positioning accuracy of the treatment part 210.

In addition, in a case where the first sheath 24 has a two-layer structure, for example, the stiffness of the first tubular member 241 against a force in the axial direction is set to be higher than stiffness of the second tubular member 242 against a force in the axial direction, and thereby accurate positioning of the distal end of the treatment part 210 is possible while lowering manufacturing costs of the first sheath 24.

In addition, it is preferable that the first sheath 24 has higher stiffness against a force in the first direction than stiffness of the second sheath 25. In this manner, manufacturing costs of the second sheath 25 can be lowered.

As described above, by making mechanical properties of the first sheath 24 and the second sheath 25 different from each other, positioning accuracy of the treatment part 210 can be improved and manufacturing costs can be optimized. In a case where the first sheath 24 and the second sheath 25 are required to have insulating properties in the endoscopic treatment equipment 20, an insulating member such as a heat-shrinkable tube may be further provided on an outer periphery thereof.

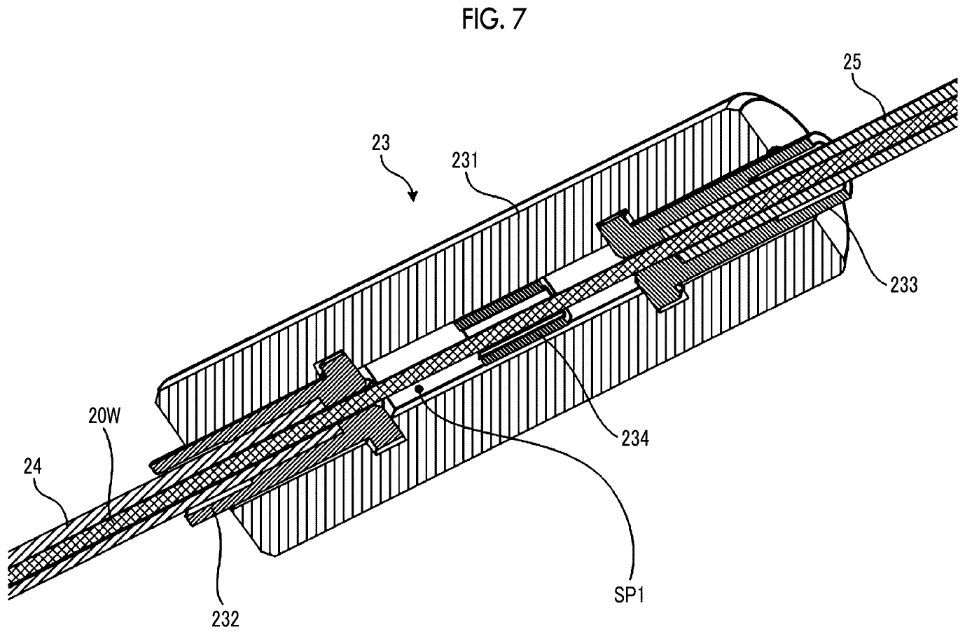

FIG. 7 is a schematic cross-sectional view of a vicinity of the operation handle 23 in the endoscopic treatment equipment 20. The operation handle 23 comprises a cylindrical support member 231 that supports the first sheath 24 and the second sheath 25 to be movable rotationally in the first direction and that forms an inner space SP1 where the operation wire 20W is disposed in the example of the figure, and a flat pipe 234 that is fixed to the operation wire 20W in the inner space SP1 and that is configured to be rotationally moved in the first direction in conjunction with the rotational movement of the support member 231 in the first direction. The flat pipe 234 is fixed to the operation wire 20W by being tightened in a state where the operation wire 20W is inserted therein or by brazing or the like. The flat pipe 234 constitutes a fixing member.

A proximal end portion of the first sheath 24 is fixed into a flanged cylindrical first sheath holder 232. The support member 231 supports the first sheath holder 232 to be movable rotationally in the first direction in the inner space SP1 and thus supports the first sheath 24 to be movable rotationally in the first direction.

A distal end portion of the second sheath 25 is fixed into a flanged cylindrical second sheath holder 233. The support member 231 supports the second sheath holder 233 to be movable rotationally in the first direction in the inner space SP1 and thus supports the second sheath 25 to be movable rotationally in the first direction.

The operation wire 20W inserted from the connector portion 22 into the second sheath 25 is inserted from the distal end of the second sheath 25 into the second sheath holder 233, is inserted from the second sheath holder 233 into the flat pipe 234, is inserted from the flat pipe 234 into the first sheath 24 via the first sheath holder 232, and is provided to extend to the distal end portion 21.

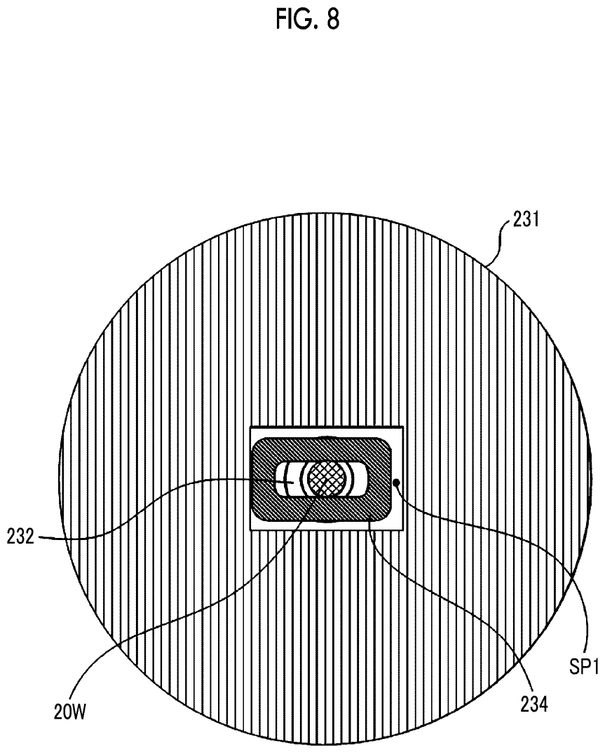

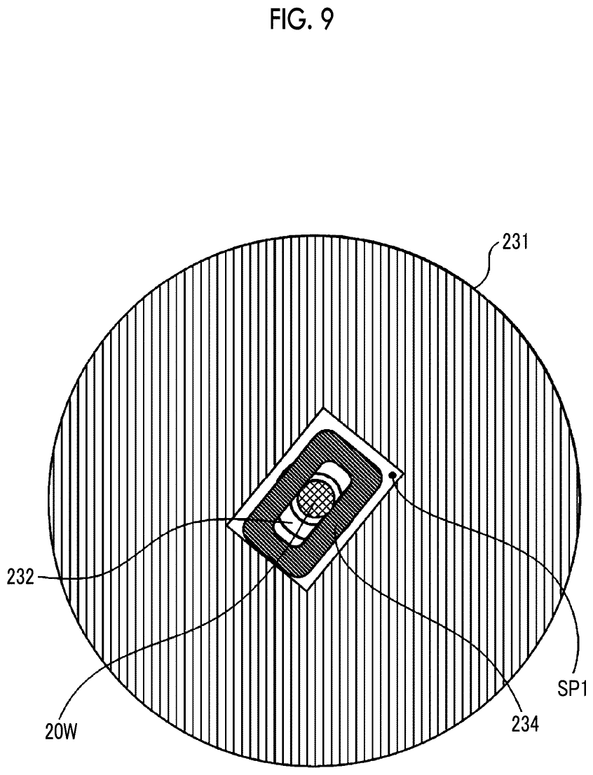

In the inner space SP1, a cross section of a portion between the first sheath holder 232 and the second sheath holder 233 perpendicular to the axial direction is rectangular. FIG. 8 is a schematic view of a cross section of the operation handle 23 perpendicular to the axial direction and is a schematic view of a cross section through the flat pipe 234. As shown in FIG. 8, the flat pipe 234 is disposed in the inner space SP1 having a rectangular cross section. The outer shape of the cross section of the flat pipe 234 is the same as that of the inner space SP1, and a gap between the flat pipe 234 and the support member 231 is small. For this reason, as shown in FIG. 9, in a case where the support member 231 is rotationally moved in the first direction, the flat pipe 234 is also rotationally moved in conjunction with the support member 231.

Since the flat pipe 234 is fixed to the operation wire 20W, the operation wire 20W is rotationally moved by the rotational movement of the support member 231, and the treatment part 210 connected to the distal end of the operation wire 20W is rotationally moved. The support member 231 supports the first sheath holder 232 and the second sheath holder 233 to be rotationally movable. For this reason, even in a case where the support member 231 is rotationally moved, the first sheath 24 and the second sheath 25 are not twisted, and an effect on a treatment is prevented.

The flat pipe 234 is movable in the axial direction between the first sheath holder 232 and the second sheath holder 233. Therefore, in a case where the slider 223 of the connector portion 22 is moved in the axial direction, the flat pipe 234 also moves between the first sheath holder 232 and the second sheath holder 233 together with the operation wire 20W. A distance between the first sheath holder 232 and the second sheath holder 233 is set to a value required for opening and closing the pair of forceps pieces 210A.

In the endoscopic treatment equipment 20 configured as described above, since the first sheath 24 and the second sheath 25 are separated from each other and are contactless, mechanical connection between the operation wire 20W and the operation handle 23 can be performed using a space therebetween without the sheaths. Accordingly, the operation wire 20W can be rotationally moved at a place different from the connector portion 22, and adjustment of the orientation of the treatment part 210 and a treatment by the treatment part 210 can be performed at different positions. As a result, two persons including the operator and the assistant can use the endoscope together. In addition, since an operation by the operator and an operation by the assistant can be performed independently of each other, the efficiency and accuracy of work can be improved.

The operation wire 20W may be configured to be divided into a first wire provided inside the first sheath 24 and a second wire provided inside the second sheath 25 and to connect the first wire and the second wire with the flat pipe 234 at the operation handle 23. The first wire constitutes a first constituent member, and the second wire constitutes a second constituent member.

In such a case, it is preferable that stiffness of the first wire against a force in the first direction and stiffness of the second wire against a force in the first direction are different from each other. Specifically, the stiffness of the second wire against the force in the first direction is lower than the stiffness of the first wire against the force in the first direction. In this manner, even in a configuration where the second wire and the connector portion 22 are fixed to be not movable rotationally relative to each other in the first direction, the rotational movement force of the operation handle 23 is unlikely to be transmitted from the second wire to the connector portion 22, and the connector portion 22 can be prevented from being moved rotationally by the rotational movement of the operation handle 23. In addition, by increasing the stiffness of the first wire against the force in the first direction, the rotational movement force of the operation handle 23 can be efficiently transmitted to the treatment part 210, and followability of the rotational movement of the treatment part 210 with respect to a rotational movement operation of the operation handle 23 can be improved.

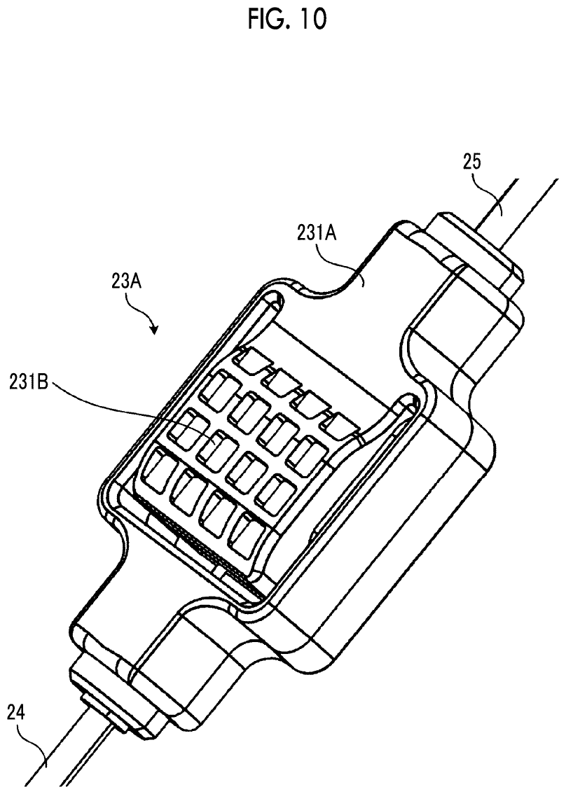

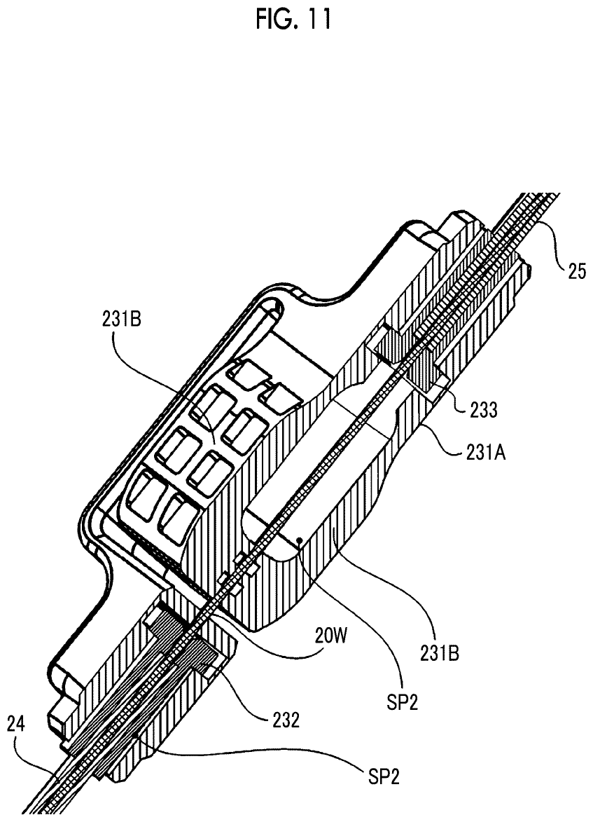

FIG. 10 is a perspective view showing a configuration of an operation handle 23A that is a first modification example of the operation handle 23. FIG. 11 is a schematic cross-sectional view of the operation handle 23A shown in FIG. 10.

The operation handle 23A has a support member 231A that is configured by joining a pair of members having the same shape and a flat shape. An inner space SP2 capable of accommodating the first sheath holder 232, the second sheath holder 233, and the operation wire 20W exposed from the first sheath holder 232 and the second sheath holder 233 is formed between the pair of members constituting the support member 231A. The support member 231A supports the first sheath holder 232 to be movable rotationally in the first direction and supports the second sheath holder 233 to be movable rotationally in the first direction. The support member 231A is provided with a pair of pressing units 231B that are elastically displaceable to face each other.

The operation wire 20W is exposed between the first sheath holder 232 and the second sheath holder 233, and the exposed portion of the operation wire 20W is disposed between the pair of pressing units 231B. A gap is provided between the pair of pressing units 231B so that the operation wire 20W is movable in the axial direction in a case where the pressing units 231B are in a steady state. In a pressed state where each pressing unit 231B is pressed in a direction of approaching the operation wire 20W, the gap disappears, the operation wire 20W is sandwiched between the pair of pressing units 231B, and the operation wire 20W cannot move in the axial direction.

By rotating the support member 231A in the first direction while maintaining the pressed state, the operation wire 20W can be moved rotationally in the first direction to rotationally move the treatment part 210. In the steady state, the operation wire 20W moves in the support member 231A in accordance with the movement of the slider 223. Therefore, an opening and closing operation of the forceps pieces 210A is possible. In addition, in this steady state, the support member 231A is rotationally movable with respect to the operation wire 20W in the first direction. Since the position of the support member 231A in the axial direction is fixed to the first sheath holder 232 and the second sheath holder 233, the operation wire 20W, the first sheath holder 232, the first sheath 24, the second sheath holder 233, and the second sheath 25 can be integrally moved in the axial direction by moving the support member 231A in the axial direction regardless of the steady state or the pressed state, and the treatment part 210 can be moved in the axial direction. As described above, the support member 231A is configured to be switched between a state where the rotational movement of the support member 231A is in conjunction with the rotational movement of the operation wire 20W and a state where the support member 231A is rotationally moved with respect to the operation wire 20W.

Since the fixing member (flat pipe) is not required in the operation handle 23A shown in FIGS. 10 and 11, restriction on a moving distance of the operation wire 20W in the axial direction in accordance with an operation of the slider 223 can be eliminated. In the operation handle 23A, a distance between the first sheath holder 232 and the second sheath holder 233 can be kept to a minimum required distance to the extent that a part of the pressing units 231B can be disposed. For this reason, even with the endoscopic treatment equipment in which the moving distance of the operation wire 20W in the axial direction in a case where the slider 223 is operated, such as a case where the treatment part 210 is composed of a snare wire, is large, the size of the operation handle 23A in the axial direction can be prevented from increasing. Accordingly, manufacturing costs of the endoscopic treatment equipment 20 can be reduced, the operability of the operation handle 23A can be improved, and the like.

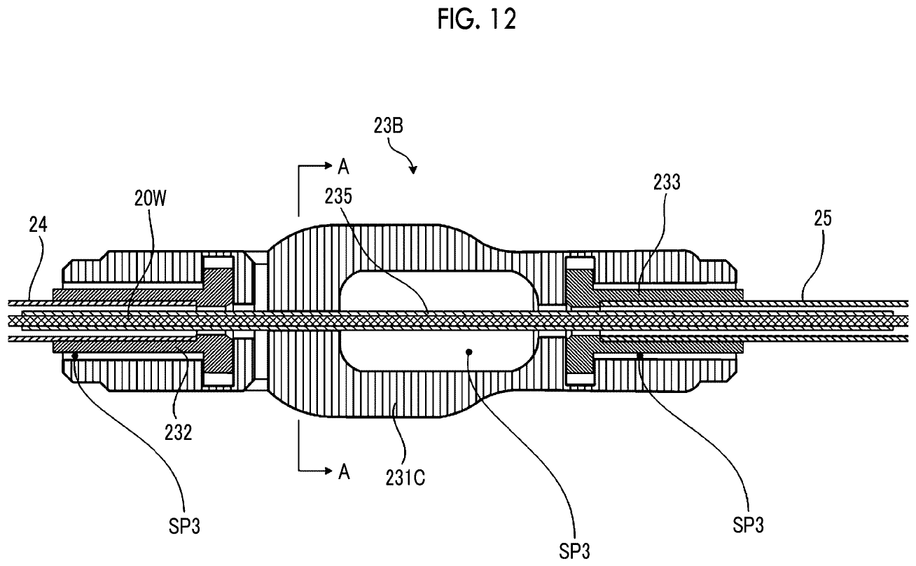

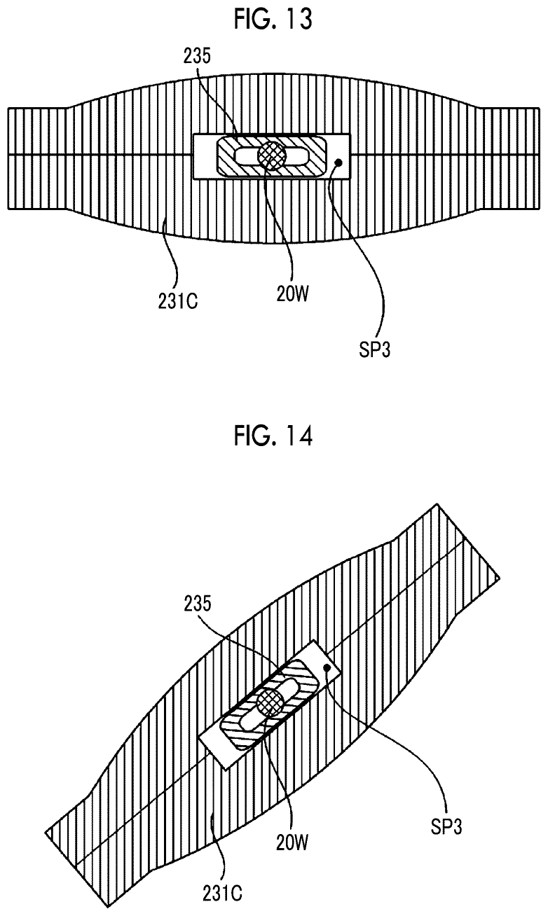

FIG. 12 is a schematic cross-sectional view showing a configuration of an operation handle 23B that is a second modification example of the operation handle 23. FIG. 13 is a schematic cross-sectional view taken along line A-A of FIG. 12.

The operation handle 23B has a support member 231C that is configured by joining a pair of members having the same shape and a flat shape. An inner space SP3 that accommodates the first sheath holder 232, the second sheath holder 233, the operation wire 20W positioned between the first sheath holder 232 and the second sheath holder 233, and an irregularly shaped tube 235 is formed between the pair of members constituting the support member 231C.

The support member 231C supports the first sheath holder 232 to be movable rotationally in the first direction and supports the second sheath holder 233 to be movable rotationally in the first direction. The operation wire 20W is inserted into the irregularly shaped tube 235, and the irregularly shaped tube 235 is fixed to the operation wire 20W. The irregularly shaped tube 235 constitutes a tubular accommodation member that accommodates the operation wire 20W.

The irregularly shaped tube 235 is provided to extend from the inside of the first sheath 24 to the inside of the second sheath 25. The irregularly shaped tube 235 is configured to have a size that allows the irregularly shaped tube 235 to be movable rotationally in the first direction in the first sheath 24 and the second sheath 25.

A portion of the irregularly shaped tube 235 between the first sheath holder 232 and the second sheath holder 233 is disposed between the pair of members constituting the support member 231C. A distance between the pair of members is a size at which the irregularly shaped tube 235 is movable in the axial direction, but is a size at which the irregularly shaped tube 235 is not movable rotationally in the first direction.

With such a configuration, as shown in FIG. 14, in a case where the support member 231C is rotationally moved in the first direction, the irregularly shaped tube 235 and the operation wire 20W are moved rotationally in the first direction in conjunction with the rotational movement. Since the irregularly shaped tube 235 can be moved rotationally in the first sheath 24 and the second sheath 25, the irregularly shaped tube 235 does not prevent the rotational movement of the support member 231C. In addition, in a case where the slider 223 is moved in the axial direction, the operation wire 20W and the irregularly shaped tube 235 accommodating the operation wire 20W are integrally moved in the axial direction, so that the forceps pieces 210A are opened and closed.

The irregularly shaped tube 235 may have a sufficient length in the axial direction to be disposed between the pair of members constituting the support member 231C regardless of the position of the slider 223 in the axial direction. In the operation handle 23B, even in a case where a distance between the first sheath 24 and the second sheath 25 is shortened, the length of the irregularly shaped tube 235 has no restriction, and the length thereof can be sufficiently increased. For this reason, as in the operation handle 23A, restriction on the moving distance of the operation wire 20W in the axial direction in accordance with an operation of the slider 223 can be eliminated while making it possible for the operation wire 20W to move in conjunction with the rotational movement of the support member 231C.

In FIG. 12, the irregularly shaped tube 235 may be configured not to be inserted into the first sheath 24. Alternatively, the irregularly shaped tube 235 may be configured not to be inserted into the second sheath 25.

In each of the endoscopic treatment equipment 20 including the operation handle 23, the endoscopic treatment equipment 20 including the operation handle 23A, and the endoscopic treatment equipment 20 including the operation handle 23B, the second sheath 25 and the operation handles 23, 23A, and 23B may be configured to be fixed, and the second sheath 25 and the sheath support portion 221 of the connector portion 22 may be configured to be connected to be movable rotationally relative to each other in the first direction.

In such a case, in a case where the operation handles 23, 23A, and 23B are rotationally moved, the second sheath 25 follows the rotational movement. However, since the second sheath 25 and the sheath support portion 221 are connected to be movable rotationally relative to each other, rotational movement forces of the operation handles 23, 23A, and 23B can be prevented from being transmitted to the connector portion 22.

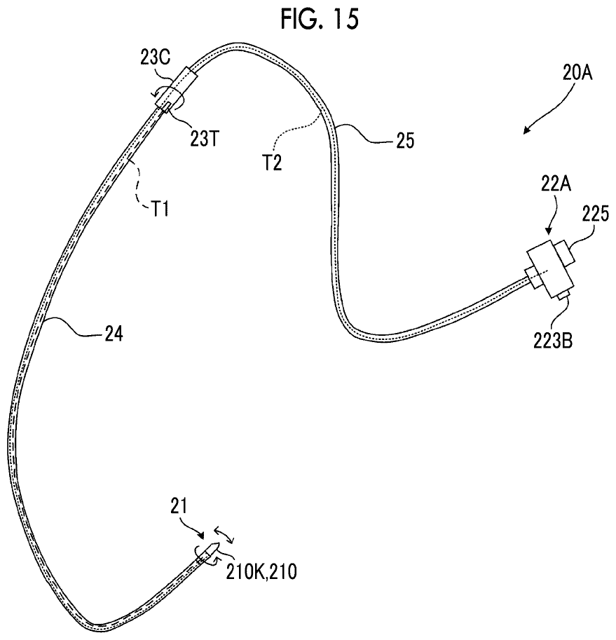

FIG. 15 is a schematic view showing a configuration of endoscopic treatment equipment 20A which is a first modification example of the endoscopic treatment equipment 20. The endoscopic treatment equipment 20A has a configuration where the operation handle 23 is changed to an operation handle 23C, the connector portion 22 is changed to a connector portion 22A, the treatment part 210 is changed to a treatment part composed of a blade-shaped high-frequency knife 210K, and the operation wire 20W is removed, compared to the endoscopic treatment equipment 20 shown in FIG. 2. For example, in FIG. 6, the distal end portion 21 of the endoscopic treatment equipment 20A has a configuration where the high-frequency knife 210K is supported by the distal end support member 211 to be movable rotationally in the first direction.

The operation handle 23C has the same configuration as that of the operation handle 23 shown in FIG. 7, but a wire support portion 23T is provided instead of the flat pipe 234. The operation handle 23C supports the proximal end portion of the first sheath 24 to be movable rotationally in the first direction and supports the distal end portion of the second sheath 25 to be movable rotationally in the first direction. The position of the operation handle 23C is fixed to the first sheath 24 and the second sheath 25 in the axial direction.

In the endoscopic treatment equipment 20A, a rotational operation wire T1 that has a distal end fixed to the high-frequency knife 210K and a proximal end fixed to the wire support portion 23T inside the operation handle 23C is provided inside the first sheath 24. The rotational operation wire T1 is a power transmission element that mechanically connects the operation handle 23C and the high-frequency knife 210K. The rotational operation wire T1 is provided inside the first sheath 24 to be movable rotationally in the first direction.

In a case where the operation handle 23C is rotationally moved in the first direction, the wire support portion 23T configured integrally with the operation handle 23C moves rotationally in the first direction, and the rotational operation wire T1 moves rotationally in conjunction with the rotational movement. As a result, the high-frequency knife 210K fixed to the rotational operation wire T1 moves rotationally in the first direction.

As described above, in the endoscopic treatment equipment 20A, a rotational movement force in a case where the operation handle 23C is rotationally moved is configured to be transmitted to the high-frequency knife 210K constituting the treatment part 210 via the rotational operation wire T1 provided only inside the first sheath 24, among the first sheath 24 and the second sheath 25.

The connector portion 22A has, for example, a configuration where the slider 223 and the ring 224 are eliminated from the configuration of the connector portion 22 shown in FIG. 4, and an operation button 225 for turning on and off supply of a high-frequency current to the high-frequency knife 210K is provided instead. In the endoscopic treatment equipment 20A, an electric wire T2 that extends from the connector portion 22A to the high-frequency knife 210K and that electrically connects the connector portion 22A and the high-frequency knife 210K is provided inside the first sheath 24, the operation handle 23C, and the second sheath 25. The electric wire T2 is a current transmission element provided to extend from the connector portion 22A to the high-frequency knife 210K. The electric wire T2 is electrically connected to a cord connected to the cord coupling part 223B. The supply of the high-frequency current to the high-frequency knife 210K is performed through the electric wire T2 in accordance with an operation of the operation button 225.

As described above, with the endoscopic treatment equipment 20A, even in a configuration where the treatment part 210 which only moves rotationally without the need for a movable portion (a forceps piece, a snare wire, or the like) in the distal end portion 21 is provided, the operator can perform a rotational movement operation and an advancing and retracting operation of the treatment part 210 (high-frequency knife 210K) using the operation handle 23C. The supply of the high-frequency current to the high-frequency knife 210K can be performed as the assistant operates the connector portion 22A. The high-frequency knife 210K can be rotationally moved at a position different from the connector portion 22A, and adjustment of the orientation of the high-frequency knife 210K and a treatment by the high-frequency knife 210K can be performed at different positions. As a result, two persons including the operator and the assistant can use the endoscope together. In addition, since an operation by the operator and an operation by the assistant can be performed independently of each other, the efficiency and accuracy of work can be improved.

In the endoscopic treatment equipment 20A, the operation handle 23C supports the second sheath 25 to be movable rotationally in the first direction, but the present disclosure is not limited thereto. For example, the operation handle 23C and the second sheath 25 may be fixed to each other. However, with a configuration where the operation handle 23C and the second sheath 25 are connected to be movable rotationally relative to each other, the second sheath 25 can be prevented from being twisted by a rotational movement operation of the operation handle 23C.

In the endoscopic treatment equipment 20A, it is preferable that the second sheath 25 and the connector portion 22A are connected to be movable rotationally relative to each other in the first direction. By doing so, for example, even in a case where the operation handle 23C and the second sheath 25 are fixed to each other, the second sheath 25 can be moved rotationally with respect to the connector portion 22A. Therefore, a rotational movement operation of the operation handle 23C can be prevented from being transmitted to the connector portion 22A.

In the endoscopic treatment equipment 20A, since it is required to accommodate the rotational operation wire T1 to be rotationally movable, it is preferable that the first sheath 24 has a tubular shape in which a hollow portion that allows the rotational operation wire T1 to be rotationally movable is provided. However, the second sheath 25 does not need to be tubular since the second sheath 25 only needs to accommodate the electric wire T2 that does not need to be moved in the axial direction and the first direction. The second sheath 25 may be composed of, for example, a solid structure in which the electric wire T2 is integrally formed by being coated with an insulating material. Accordingly, the costs can be further reduced.

Also in the endoscopic treatment equipment 20A, as in the endoscopic treatment equipment 20, by making mechanical properties (stiffness against a force in the first direction or the axial direction) different between the first sheath 24 and the second sheath 25, manufacturing costs can be optimized, operability can be improved, and the like.

For example, by making stiffness of the first sheath 24 against a force in the first direction higher than stiffness of the second sheath 25 against a force in the first direction, a reaction force of the second sheath 25 caused by twisting of the second sheath 25 can be suppressed in a case where the operation handle 23C and the second sheath 25 are fixed to each other. In addition, in a case where the operation handle 23C and the second sheath 25 are fixed to each other and the second sheath 25 and the connector portion 22A are fixed to each other, a rotational movement force of the operation handle 23C can be prevented from being transmitted to the connector portion 22A. In addition, costs of the second sheath 25 can be reduced.

In addition, by making stiffness of the first sheath 24 against a force in the axial direction lower than stiffness of the second sheath 25 against a force in the axial direction, costs of the second sheath 25 can be lowered while the high-frequency knife 210K can be positioned with high accuracy.

In the endoscopic treatment equipment 20A, the high-frequency knife 210K may be replaced with, for example, an injection needle. In this case, a chemical liquid transmission element may be provided instead of the electric wire T2, and a cylinder for chemical liquid injection may be provided at the connector portion 22A. In addition, in some treatment tools, both the chemical liquid transmission element and the current transmission element (electric wire T2) may be provided to extend from the connector portion 22A to the treatment part 210. In this case, for example, the operation button 225 may be removed from the connector portion 22A in FIG. 15, only the cord coupling part 223B and a chemical liquid injection port may be provided, the assistant may perform only chemical liquid injection work, and an electrical connection operation may be performed by the operator using a foot switch or the like (not shown). In the treatment tool having a plurality of operation degrees of freedom in this way, various combinations are possible as to which operation unit is disposed at the connector portion. The examples do not limit the combinations. In addition, in FIG. 15, in a case where the electric wire T2 is composed of at least one of the chemical liquid transmission element or the current transmission element, the second sheath 25 does not need to have a tubular shape having a hollow portion, and costs can be lowered by adopting a solid structure.

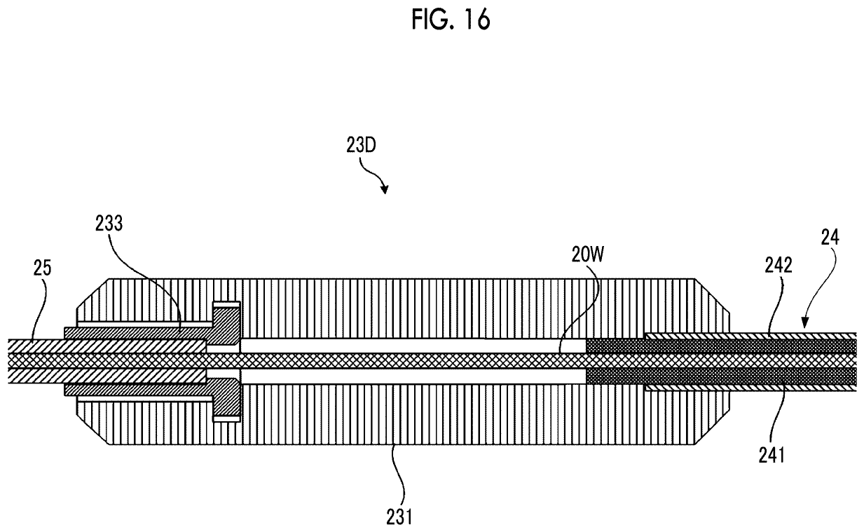

FIG. 16 is a schematic cross-sectional view showing a configuration of an operation handle 23D that is a third modification example of the operation handle 23 of the endoscopic treatment equipment 20. The operation handle 23D is different from the configuration of the operation handle 23 shown in FIG. 7 in that the flat pipe 234 and the first sheath holder 232 are removed, and the first sheath 24 and the support member 231 are fixed to each other by adhesion or the like so that the first sheath 24 and the support member 231 are not movable rotationally relative to each other. In addition, in the endoscopic treatment equipment 20 in which the operation handle 23 is substituted with the operation handle 23D (hereinafter, referred to as the endoscopic treatment equipment 20D), the link mechanism support member 210D and the distal end support member 211 shown in FIG. 6 are configured to be integrated with each other.

In the endoscopic treatment equipment 20D, the first tubular member 241 constituting an inner layer of the first sheath 24 protrudes from a proximal end edge of the second tubular member 242, and the protruding portion is adhered to, for example, an inner peripheral surface of the support member 231. In addition, for example, a proximal end portion of the second tubular member 242 constituting an outer layer of the first sheath 24 is adhered to the inner peripheral surface of the support member 231.

As described above, in the endoscopic treatment equipment 20D, since the support member 231 and the first sheath 24 are fixed to each other, in a case where the support member 231 is moved rotationally in the first direction, the first sheath 24 is moved rotationally in conjunction with the rotational movement. As a result, the distal end support member 211 that supports the distal end portion of the first sheath 24 and the treatment part 210 supported by the distal end support member 211 are rotationally moved. As described above, in the endoscopic treatment equipment 20D including the operation handle 23D, the support member 231 and the treatment part 210 are mechanically connected by the first sheath 24, and a rotational movement force in a case where the support member 231 is rotationally moved is configured to be transmitted to the treatment part 210 via the first sheath 24.

In the endoscopic treatment equipment 20D, the first sheath 24 transmits a rotational movement force of the support member 231 to the treatment part 210. Therefore, it is preferable that the first sheath 24 has high stiffness against a force in the first direction. For example, it is preferable that the first sheath 24 has higher stiffness against the force in the first direction than stiffness of the second sheath 25. Accordingly, followability of the rotational movement of the treatment part 210 caused by a rotational movement operation of the operation handle 23D can be improved while reducing the costs of the second sheath 25. In addition, it is preferable that the first sheath 24 has higher stiffness against a force in the axial direction than stiffness of the second sheath 25. Accordingly, positioning accuracy of the treatment part 210 and a grasping force of the forceps pieces 210A can be sufficiently ensured while reducing the costs of the second sheath 25.

Also in the endoscopic treatment equipment 20D, the second sheath 25 and the connector portion 22 may be connected to be movable rotationally relative to each other in the first direction. The support member 231 is movable rotationally with respect to the second sheath 25, but the second sheath 25 is movable rotationally with respect to the connector portion 22 as well. Therefore, an operation of the connector portion 22 can be more effectively prevented from affecting an operation of the operation handle 23D.

In the endoscopic treatment equipment 20D, in addition to the operation wire 20W, one or both of the current transmission element and the chemical liquid transmission element extending from the connector portion 22 to the treatment part 210 may be provided inside the first sheath 24, the operation handle 23D, and the second sheath 25.

In the endoscopic treatment equipment 20D, in a case where the treatment part 210 does not have a movable portion and a current needs to be supplied to the treatment part 210 (for example, in a case of the high-frequency knife 210K shown in FIG. 15), for example, a current transmission element may be provided instead of the operation wire 20W. In addition, in the endoscopic treatment equipment 20D, in a case where the treatment part 210 is an injection needle, for example, a chemical liquid transmission element may be provided instead of the operation wire 20W. In the endoscopic treatment equipment 20D, in a case where the operation wire 20W is substituted with one or both of the current transmission element and the chemical liquid transmission element, at least one of the first sheath 24 or the second sheath 25 does not need to have a hollow tubular internal structure and may have a solid structure in which the current transmission element and the chemical liquid transmission element are integrally provided.

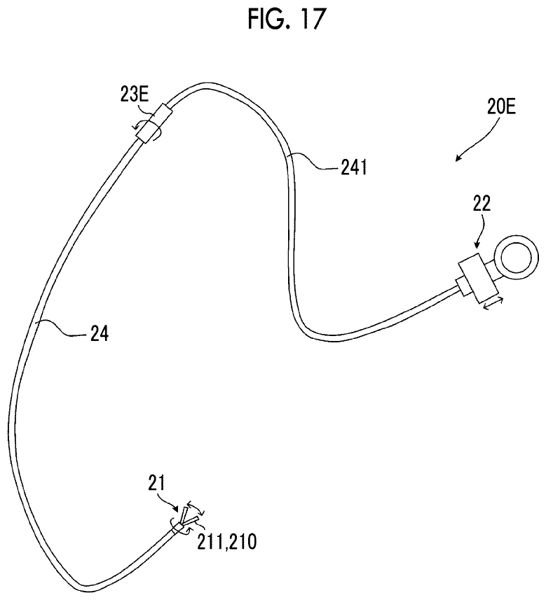

FIG. 17 is a schematic view showing a configuration of endoscopic treatment equipment 20E which is a second modification example of the endoscopic treatment equipment 20. The endoscopic treatment equipment 20E has a configuration where the operation handle 23 is changed to an operation handle 23E, the second sheath 25 is removed, and the first tubular member 241 constituting the inner layer of the first sheath 24 is extended to the position of the connector portion 22 instead of the removed second sheath 25, compared to the endoscopic treatment equipment 20 shown in FIG. 2.

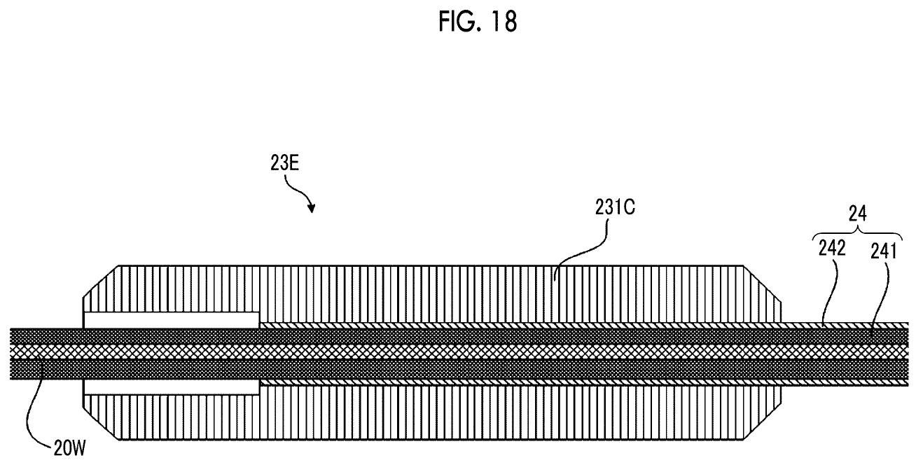

FIG. 18 is a schematic view showing a cross section of a vicinity of the operation handle 23E of the endoscopic treatment equipment 20E shown in FIG. 17. In FIG. 18, a left side is a proximal end side of the endoscopic treatment equipment 20E, and a right side is a distal end side of the endoscopic treatment equipment 20E. In the endoscopic treatment equipment 20E, the link mechanism support member 210D and the distal end support member 211 shown in FIG. 6 are configured to be integrated with each other.

The operation handle 23E comprises the cylindrical support member 231C. The second tubular member 242 is fixed to an inner peripheral surface of the support member 231C by adhesion or the like. In the first sheath 24, the first tubular member 241 and the second tubular member 242 are configured to be rotationally moved relative to each other in the first direction. The first tubular member 241 and the second tubular member 242 are configured not to be moved relative to each other in the axial direction at least at the distal end portion 21. That is, the position of the second tubular member 242 in the axial direction is fixed with respect to the first tubular member 241. As shown in FIG. 6, a distal end portion of the second tubular member 242 of the first sheath 24 is fixed to the distal end support member 211.

The first sheath 24 has a two-layer structure which is changed to a one-layer structure with the inside of the support member 231C as a boundary. Specifically, the second tubular member 242 extends from the distal end portion 21 to the inside of the support member 231C, and the first tubular member 241 protrudes from the proximal end edge of the second tubular member 242. The first tubular member 241 extends to the inside of the sheath support portion 221 of the connector portion 22 shown in FIG. 4 and is supported by the sheath support portion 221. In a case where the endoscopic treatment equipment 20E is required to have insulating properties, the second tubular member 242 exposed on the proximal end side of the operation handle 23E is coated with an insulating member such as a heat-shrinkable tube.

In the endoscopic treatment equipment 20E, since the support member 231C and the second tubular member 242 are fixed, in a case where the support member 231C is rotationally moved in the first direction, the second tubular member 242 is rotationally moved. As a result, the distal end support member 211 that supports the distal end portion of the second tubular member 242 and the treatment part 210 supported by the distal end support member 211 are rotationally moved. As described above, in the endoscopic treatment equipment 20E, the support member 231C and the treatment part 210 are mechanically connected by the first sheath 24, and a rotational movement force in a case where the support member 231C is rotationally moved is configured to be transmitted to the treatment part 210 via the first sheath 24. In a case where the support member 231 is moved in the axial direction, the first sheath 24 and the operation handle 23E are integrally moved in the axial direction, so that the position of the treatment part 210 in the axial direction can be changed. In the endoscopic treatment equipment 20E, the first tubular member 241 constitutes a first member. The second tubular member 242 constitutes a tubular member.

As described above, with the endoscopic treatment equipment 20E, the operator can perform a rotational movement operation and an advancing and retracting operation of the treatment part 210 using the operation handle 23E. For this reason, the same effects as those of the endoscopic treatment equipment 20 are obtained. With the operation handle 23E, since the second sheath 25 is not required, manufacturing costs can be lowered.

In the endoscopic treatment equipment 20E, it is preferable that the first tubular member 241 and the second tubular member 242 have different mechanical properties. For example, it is preferable that the second tubular member 242 has higher stiffness against a force in the first direction than stiffness of the first tubular member 241. In this manner, followability of the rotational movement of the treatment part 210 with respect to a rotational movement operation of the operation handle 23E can be improved while lowering manufacturing costs of the first sheath 24. In addition, it is preferable that the first tubular member 241 has higher stiffness against a force in the axial direction than stiffness of the second tubular member 242. In this manner, positioning accuracy of the treatment part 210 and a grasping force of the forceps pieces 210A can be sufficiently ensured.

In the endoscopic treatment equipment 20E, the operation handle 23E is not essential and can be omitted. In a case where the operation handle 23E is omitted, the operator pinches the proximal end portion of the second tubular member 242 with his or her fingers to move the second tubular member 242 rotationally in the first direction or to move the second tubular member 242 in the axial direction, so that the treatment part 210 can move rotationally and advance and retract.

In the endoscopic treatment equipment 20E, the first tubular member 241 may be fixed to the inner peripheral surface of the support member 231C, and the second tubular member 242 and the first tubular member 241 may be not movable rotationally relative to each other in the first direction. In this case, in a case where the operation handle 23E is rotationally moved, the entire first sheath 24 is rotationally moved with respect to the operation wire 20W, so that the treatment part 210 can be rotationally moved. In this case, a rotational movement force in a case where the operation handle 23E is rotationally moved can be transmitted to the connector portion 22 via the first tubular member 241. However, by making stiffness of the first tubular member 241 against a force in the first direction lower than the stiffness of the second tubular member 242 against a force in the first direction, a rotational movement force of the support member 231C can be unlikely to be transmitted to the connector portion 22. As a result, a reaction force caused by twisting of the exposed first tubular member 241 is suppressed, and a rotational movement operation of the operation handle 23E by the operator can be prevented from affecting an operation of the connector portion 22 by the assistant.

In addition, in a configuration where the second tubular member 242 and the first tubular member 241 are not movable rotationally relative to each other in the support member 231C, for example, even in a case where mechanical properties of the first tubular member 241 and the second tubular member 242 are the same, a transmission performance of a rotational movement force of the operation handle 23E is changed between a portion having the two-layer structure and a portion having the one-layer structure by changing from the two-layer structure to the one layer structure of the first sheath 24 with the operation handle 23E as a boundary. For this reason, it is possible to prevent a rotational movement operation of the operation handle 23E by the operator from affecting an operation of the connector portion 22 by the assistant. In addition, such an effect can be eliminated by connecting the first tubular member 241 and the sheath support portion 221 to be movable rotationally relative to each other in the first direction.

In the endoscopic treatment equipment 20E, instead of a single member constituting the first tubular member 241, the first tubular member 241 may be divided into two members in the axial direction, and the two members may be configured to be coupled to each other at the support member 231C. For example, a coupling part of the two members constituting the first tubular member 241 is provided inside the support member 231C on the proximal end side, and a member on the proximal end side of the coupling part is formed of a resin tube or the like, and thereby costs can be reduced.

In the endoscopic treatment equipment 20E, in addition to the operation wire 20W, at least one of a current transmission element or a chemical liquid transmission element may be further provided in some cases. In addition, instead of the operation wire 20W, at least one of the current transmission element or the chemical liquid transmission element may be provided in some cases. In a case where at least one of the current transmission element or the chemical liquid transmission element is provided instead of the operation wire 20W, the first tubular member 241 does not need to have a tubular shape having a hollow portion and may be a solid structure in which at least one of the transmission element or the chemical liquid transmission element is integrally provided inside.

As described hereinbefore, at least the following matters are described in the present specification.

(1)

An endoscopic treatment equipment to be used by being introduced into a treatment tool inlet port of an endoscope, the endoscopic treatment equipment comprising:
a distal end portion that includes a treatment part;
a proximal end portion that is provided at a proximal end;
an operation member that is provided between the distal end portion and the proximal end portion and that is mechanically connected to the treatment part;
a first connecting member that connects the distal end portion and the operation member; and
a second connecting member that connects the operation member and the proximal end portion and that is different from the first connecting member,
in which the operation member is configured to be rotationally moved in a first direction about an axis of the endoscopic treatment equipment, and
the treatment part is configured to be rotationally moved in the first direction by a rotational movement of the operation member.
   (2)

The endoscopic treatment equipment according to (1), p1 in which a position of the operation member in an axial direction of the endoscopic treatment equipment is fixed to the first connecting member and the second connecting member.

(3)

The endoscopic treatment equipment according to (2), further comprising:
a power transmission element that mechanically connects at least the operation member and the treatment part,
in which the operation member is configured to be rotationally moved in the first direction with respect to the first connecting member, and
the endoscopic treatment equipment is configured such that a rotational movement force in a case where the operation member is moved rotationally is transmitted to the treatment part via the power transmission element.
   (4)

The endoscopic treatment equipment according to (3),
in which the first connecting member has a tubular shape in which the power transmission element is provided.
   (5)

The endoscopic treatment equipment according to (4),
in which the power transmission element is provided to further extend from the operation member to the proximal end portion and mechanically connects the proximal end portion and the treatment part, and
the second connecting member has a tubular shape in which the power transmission element is provided.
   (6)

The endoscopic treatment equipment according to (5),
in which inside the first connecting member, the operation member and the second connecting member, the power transmission element is provided to be movable in the axial direction.
   (7)

The endoscopic treatment equipment according to (6),
in which the operation member is configured to be rotationally moved in the first direction with respect to the first connecting member and the second connecting member.
   (8)

The endoscopic treatment equipment according to (7),
in which the operation member includes a support member that supports the first connecting member and the second connecting member to be movable rotationally and that forms an inner space where the power transmission element is disposed, and a fixing member that is fixed to the power transmission element in the inner space and that is configured to be rotationally moved in conjunction with a rotational movement of the support member.
   (9)

The endoscopic treatment equipment according to (7),
in which the operation member includes a support member that supports the first connecting member and the second connecting member to be movable rotationally and that forms an inner space where the power transmission element is disposed, and the support member is configured to be switched between a state where a rotational movement of the power transmission element is in conjunction with a rotational movement of the support member and a state where the support member is moved rotationally with respect to the power transmission element.
   (10)

The endoscopic treatment equipment according to (7),
in which the operation member includes a support member that supports the first connecting member and the second connecting member to be movable rotationally and that forms an inner space where the power transmission element is disposed, and a tubular accommodation member that accommodates the power transmission element, and
the accommodation member is configured to be rotationally moved in conjunction with a rotational movement of the support member, and a part thereof is provided to be movable rotationally and movable in the axial direction inside at least one of the first connecting member or the second connecting member.
   (11)

The endoscopic treatment equipment according to (7),
in which the power transmission element and the proximal end portion are connected to be movable rotationally relative to each other in the first direction.
   (12)

The endoscopic treatment equipment according to (7),
in which the power transmission element is configured such that a first constituent member provided inside the first connecting member and a second constituent member provided inside the second connecting member are connected at the operation member, and the second constituent member has stiffness against a force in the first direction lower than stiffness of the first constituent member.
   (13)

The endoscopic treatment equipment according to (4), further comprising:
a transmission element that is provided to extend from the proximal end portion to the treatment part,
in which the second connecting member is a solid structure in which the transmission element is provided.
   (14)

The endoscopic treatment equipment according to (2),
in which the operation member is configured to be rotationally moved in the first direction with respect to the second connecting member.
   (15)

The endoscopic treatment equipment according to (2),
in which the operation member is fixed to the first connecting member, and
the endoscopic treatment equipment is configured such that a rotational movement force in a case where the operation member is moved rotationally is transmitted to the treatment part via the first connecting member.
   (16)

The endoscopic treatment equipment according to (15), further comprising:
a transmission element that is provided to extend from the proximal end portion to the treatment part,
in which the first connecting member and the second connecting member each have a columnar shape in which the transmission element is provided.
   (17)

The endoscopic treatment equipment according to (16),
in which the transmission element includes a power transmission element that mechanically connects the proximal end portion and the treatment part,
the first connecting member has a first tubular member and a second tubular member provided on an outer periphery of the first tubular member,
inside the first tubular member, the power transmission element is provided to be movable in the axial direction, and
the first tubular member has higher stiffness against a force in the axial direction than stiffness of the second tubular member against a force in the axial direction.
   (18)

The endoscopic treatment equipment according to any one of (1) to (17),
in which the first connecting member and the second connecting member have different mechanical properties.
   (19)

The endoscopic treatment equipment according to (18),
in which stiffness of the first connecting member against a force in the first direction and stiffness of the second connecting member against a force in the first direction are different.
   (20)

The endoscopic treatment equipment according to (19),
in which the first connecting member has higher stiffness against the force in the first direction than the stiffness of the second connecting member against the force in the first direction.
   (21)

The endoscopic treatment equipment according to (18),
in which stiffness of the first connecting member against a force in an axial direction of the endoscopic treatment equipment and stiffness of the second connecting member against a force in the axial direction of the endoscopic treatment equipment are different.
   (22)

The endoscopic treatment equipment according to (21),
in which the first connecting member has higher stiffness against the force in the axial direction than the stiffness of the second connecting member against the force in the axial direction.
   (23)

The endoscopic treatment equipment according to any one of (1) to (17),
in which the proximal end portion and the second connecting member are connected to be movable rotationally relative to each other.
   (24)

An endoscopic treatment equipment to be used by being introduced into a treatment tool inlet port of an endoscope, the endoscopic treatment equipment comprising:
a distal end portion that includes a treatment part;
a proximal end portion that is provided at a proximal end of the endoscopic treatment equipment;
a first member that extends from the distal end portion to the proximal end portion along an axial direction of the endoscopic treatment equipment; and
a tubular member in which a part of the first member is provided and that extends from the distal end portion to a position between the distal end portion and the proximal end portion,
in which the treatment part is configured to be rotationally moved in a first direction about an axis of the endoscopic treatment equipment by a rotational movement of the tubular member in the first direction.
   (25)

The endoscopic treatment equipment according to (24),
in which the tubular member is configured to be rotationally moved in the first direction with respect to the first member.
   (26)

The endoscopic treatment equipment according to (24),
in which at least at the distal end portion, a position of the tubular member in the axial direction of the endoscopic treatment equipment is fixed to the first member.
   (27)

The endoscopic treatment equipment according to any one of (24) to (26), further comprising:
an operation member that is fixed to the tubular member.
   (28)

The endoscopic treatment equipment according to any one of (24) to (26), further comprising:
a transmission element that is provided to extend from the proximal end portion to the treatment part.
   (29)

The endoscopic treatment equipment according to (28),
in which the first member has a tubular shape, and
inside the first member, the transmission element is provided to be movable in the axial direction.
   (30)

The endoscopic treatment equipment according to (28),
in which the first member is a solid structure in which the transmission element is provided.
   (31)

The endoscopic treatment equipment according to any one of (24) to (26),
in which the first member and the tubular member have different mechanical properties.
   (32)

The endoscopic treatment equipment according to (31),
in which stiffness of the first member against a force in the first direction and stiffness of the tubular member against a force in the first direction are different.
   (33)

The endoscopic treatment equipment according to (32),
in which the tubular member has higher stiffness against the force in the first direction than the stiffness of the first member against the force in the first direction.
   (34)

The endoscopic treatment equipment according to any one of (24) to (26),
in which stiffness of the first member against a force in the axial direction of the endoscopic treatment equipment and stiffness of the tubular member against a force in the axial direction of the endoscopic treatment equipment are different.
   (35)

The endoscopic treatment equipment according to (34),
in which the first member has higher stiffness against the force in the axial direction than the stiffness of the tubular member against the force in the axial direction.

### EXPLANATION OF REFERENCES

1: endoscope
2: body part
T2: electric wire
4: light source device
4A: connecting portion
5: processor device
7: display device
10: insertion part
10A: soft portion
10B: bendable part
10C, 21: distal end portion
10E: distal end surface
11: operating part
11A: suction button
12: angle knob
13: universal cord
13A, 22, 22A: connector portion
20, 20A, 20D, 20E: endoscopic treatment equipment
20W: operation wire
23, 23A, 23B, 23C, 23D, 23E: operation handle
23T: wire support portion
24: first sheath
25: second sheath
100: endoscope device
112: treatment tool inlet port
210: treatment part
210A: forceps piece
210B: wire support member
210C: link mechanism
210D: link mechanism support member
210K: high-frequency knife
211: distal end support member
221: sheath support portion
222: slider shaft
223: slider
223A: flange portion
223B: cord coupling part
224: ring
225: operation button
231, 231A, 231C: support member
231B: pressing unit
232: first sheath holder
233: second sheath holder
234: flat pipe
235: irregularly shaped tube
241: first tubular member
242: second tubular member
SP1, SP2, SP3: inner space
L1, L2: distance
T1: rotational operation wire

What is claimed is:

1. An endoscopic treatment equipment to be used by being introduced into a treatment tool inlet port of an endoscope, the endoscopic treatment equipment comprising:
a distal end portion that includes a treatment part;
a proximal end portion that is provided at a proximal end of the endoscopic treatment equipment;
an operation member that is provided between the distal end portion and the proximal end portion and that is mechanically connected to the treatment part;
a first connecting member that connects the distal end portion and the operation member; and
a second connecting member that connects the operation member and the proximal end portion and that is different from the first connecting member,
wherein the operation member is configured to be rotationally moved in a first direction about an axis of the endoscopic treatment equipment, and
the treatment part is configured to be rotationally moved in the first direction by a rotational movement of the operation member.

2. The endoscopic treatment equipment according to claim 1,
wherein a position of the operation member in an axial direction of the endoscopic treatment equipment is fixed to the first connecting member and the second connecting member.

3. The endoscopic treatment equipment according to claim 2, further comprising:
a power transmission element that mechanically connects at least the operation member and the treatment part,
wherein the operation member is configured to be rotationally moved in the first direction with respect to the first connecting member, and
the endoscopic treatment equipment is configured such that a rotational movement force in a case where the operation member is moved rotationally is transmitted to the treatment part via the power transmission element.

4. The endoscopic treatment equipment according to claim 3,
wherein the first connecting member has a tubular shape in which the power transmission element is provided.

5. The endoscopic treatment equipment according to claim 4,
wherein the power transmission element is provided to further extend from the operation member to the proximal end portion and mechanically connects the proximal end portion and the treatment part, and
the second connecting member has a tubular shape in which the power transmission element is provided.

6. The endoscopic treatment equipment according to claim 5,
wherein inside the first connecting member, the operation member and the second connecting member, the power transmission element is provided to be movable in the axial direction.

7. The endoscopic treatment equipment according to claim 6,
wherein the operation member is configured to be rotationally moved in the first direction with respect to the first connecting member and the second connecting member.

8. The endoscopic treatment equipment according to claim 7,
wherein the operation member includes a support member that supports the first connecting member and the second connecting member to be movable rotationally and that forms an inner space where the power transmission element is disposed, and a fixing member that is fixed to the power transmission element in the inner space and that is configured to be rotationally moved in conjunction with a rotational movement of the support member.

9. The endoscopic treatment equipment according to claim 7,
wherein the power transmission element and the proximal end portion are connected to be movable rotationally relative to each other in the first direction.

10. The endoscopic treatment equipment according to claim 7,
wherein the power transmission element is configured such that a first constituent member provided inside the first connecting member and a second constituent member provided inside the second connecting member are connected at the operation member, and
the second constituent member has stiffness against a force in the first direction lower than stiffness of the first constituent member against a force in the first direction.

11. The endoscopic treatment equipment according to claim 4, further comprising:
a transmission element that is provided to extend from the proximal end portion to the treatment part,
wherein the second connecting member is a solid structure in which the transmission element is provided.

12. The endoscopic treatment equipment according to claim 2,
wherein the operation member is configured to be rotationally moved in the first direction with respect to the second connecting member.

13. The endoscopic treatment equipment according to claim 2,
wherein the operation member is fixed to the first connecting member, and
the endoscopic treatment equipment is configured such that a rotational movement force in a case where the operation member is moved rotationally is transmitted to the treatment part via the first connecting member.

14. The endoscopic treatment equipment according to claim 13, further comprising:
a transmission element that is provided to extend from the proximal end portion to the treatment part,
wherein the first connecting member and the second connecting member each have a columnar shape in which the transmission element is provided.

15. The endoscopic treatment equipment according to claim 14,
wherein the transmission element includes a power transmission element that mechanically connects the proximal end portion and the treatment part,
the first connecting member has a first tubular member and a second tubular member provided on an outer periphery of the first tubular member,
inside the first tubular member, the power transmission element is provided to be movable in the axial direction, and
the first tubular member has higher stiffness against a force in the axial direction than stiffness of the second tubular member against a force in the axial direction.

16. The endoscopic treatment equipment according to claim 1,
wherein stiffness of the first connecting member against a force in the first direction and stiffness of the second connecting member against a force in the first direction are different.

17. The endoscopic treatment equipment according to claim 1,
wherein stiffness of the first connecting member against a force in an axial direction of the endoscopic treatment equipment and stiffness of the second connecting member against a force in the axial direction of the endoscopic treatment equipment are different.

18. The endoscopic treatment equipment according to claim 1,
wherein the proximal end portion and the second connecting member are connected to be movable rotationally relative to each other.

19. An endoscopic treatment equipment to be used by being introduced into a treatment tool inlet port of an endoscope, the endoscopic treatment equipment comprising:
a distal end portion that includes a treatment part;
a proximal end portion that is provided at a proximal end of the endoscopic treatment equipment;
a first member that extends from the distal end portion to the proximal end portion along an axial direction of the endoscopic treatment equipment; and
a tubular member in which a part of the first member is provided and that extends from the distal end portion to a position between the distal end portion and the proximal end portion,
wherein the treatment part is configured to be rotationally moved in a first direction about an axis of the endoscopic treatment equipment by a rotational movement of the tubular member in the first direction.

20. The endoscopic treatment equipment according to claim 19,
wherein the tubular member is configured to be rotationally moved in the first direction with respect to the first member.

21. The endoscopic treatment equipment according to claim 19, further comprising:
an operation member that is fixed to the tubular member.

22. The endoscopic treatment equipment according to claim 19, further comprising:
a transmission element that is provided to extend from the proximal end portion to the treatment part.

23. The endoscopic treatment equipment according to claim 22,
wherein the first member has a tubular shape, and
inside the first member, the transmission element is provided to be movable in the axial direction.

24. The endoscopic treatment equipment according to claim 22,
wherein the first member is a solid structure in which the transmission element is provided.

25. The endoscopic treatment equipment according to claim 19,
wherein stiffness of the first member against a force in the first direction and stiffness of the tubular member against a force in the first direction are different.

26. The endoscopic treatment equipment according to claim 19,
wherein the first member has higher stiffness against the force in the axial direction than the stiffness of the tubular member against the force in the axial direction.
